(19) United States
(12) Patent Application Publication
(10) Pub. No.: US 2005/0240278 A1
(43) Pub. Date: Oct. 27, 2005

(54) Stent improvements

(76) Inventors: Peter Aliski, Newton, MA (US); Vasu Nishtala, Snellville, GA (US); Benedict Shia, Needham, MA (US); Anthony Tremaglio, Charlestown, MA (US)

Correspondence Address: GANZ LAW, P.C., P O BOX 2200, HILLSBORO, OR, 97123, US

(21) Appl. No.: 10/833,210

(22) Filed: Apr. 26, 2004

(51) Int. Cl. A61F 2/04

(52) U.S. Cl. 623/23.7; 604/8

(57) ABSTRACT

In accordance with the invention, there are provided medical devices for providing a fluid passage between two areas in the body. An embodiment of the present invention relates to a stent comprising a tubular member at least a portion of which comprises a reinforcement structure. The tubular member includes a wall and an axial lumen therein having a lumen surface. The reinforcement structure extends at least a portion of a length of the body portion adjacent the distal end portion. One advantage of this embodiment of the invention is, for example, that the reinforcement structure increases patient comfort by providing enhanced flexibility. A second advantage of this embodiment of the invention is, for example, that the reinforcement structure improves axial stiffness and radial stiffness allowing for a thinner tubular member wall thickness and larger lumen diameter.

### FIELD OF THE INVENTION

The present invention is related to ureteral stents, and more particularly, to methods and apparatus for stent shaft improvement.

### BACKGROUND

Tubular prostheses, which are commonly referred to as stents, are used in a variety of medical procedures. For example, stents are often used in connection with assisting drainage from the kidney through the ureter, from the liver through the biliary ducts, from the dorsal or ventral pancreas through the pancreatic ducts, from the gall bladder through the cystic, hepatic, or common bile ducts, and the like. A leading reason for stent deployment in ducts is to provide drainage to circumvent a blockage. Blockage of ducts in the body can be a serious and very painful affliction that can result in death if not promptly and effectively treated. Blockage can occur for a number of reasons. In the kidney, for example, stones, or debris from such stones, can pass into the ureter, where they become entrapped. Similarly, in the gall bladder, stones or debris can pass into the bile ducts, where they become entrapped. Alternatively, cysts or tumors growing against the outer wall of the ducts can cause constriction of the ducts. Similarly, internal or duct wall cysts or tumors can act to block ducts.

The main function of ureteral stents, for example, is to bypass ureteral obstruction and to provide urinary drainage from the kidney to the bladder for a period of time, typically a few days to several months. The ureteral stent is usually provided with a drainage means such as a lumen for directing fluid from the renal pelvis to the bladder. Conventional stents include openings provided along the stent for communication with the lumen to aide in drainage.

Early ureteral stents were straight. As a result, after placement into the ureter, these straight stents often migrated or were expelled from the ureter as a result of peristaltic action by the ureter. Later ureteral stents, therefore, were usually designed with means of retention on one or both ends of the stent. The retention means is intended to inhibit stent migration either upward into the kidney or downward into the bladder. Retention means that have been employed are in the form of hooks, pigtails, coils, corkscrews, malecots, barbs, mushrooms, or any other practical shape that will serve the purpose.

Current urinary stents comprise a shaft commonly made of either single or dual durometer polymer material. Current shaft designs often have unique profile cross-sections and hydrophilic or anti-microbial coatings. This shaft typically resides in the ureter to provide drainage of urine post ureteroscopy procedures. Anecdotally, it is believed that the softer the material, the less irritation to the ureter, and the greater the patient comfort. The problem with making the shaft extremely soft is that the lack of stiffness makes it difficult to push the stent into the patient. Hence for placement, a certain axial stiffness is built in which equates to a high level of radial stiffness. Such stents are believed to be felt by the muscle spasm of the ureter, potentially causing patient discomfort. Further, the axial stiffness of current stents may not be ideal for comfort to the urinary tract anatomy without being felt by the patent.

In addition to varying lengths, ureteral stents are also made with varying diameters, e.g., from 3 French (1 mm) to 16 French (5.28 mm), and typically, 4.5 French (1.5 mm) to 8.5 French (2.8 mm), and varying degrees of hardness. Ureteral stents with smaller diameters are usually easier to insert but may provide insufficient drainage, whereas stents with larger diameters allow for increasing drainage capacity through the ureter but may be difficult to insert. Stiff ureteral stents are also easier to insert than are softer stents, but once inserted can lead to increased patient discomfort. Softer stents, on the other hand, provide more comfort for the patient but are more difficult to insert due to their softness. Presently, most available stents are either made of silicone or of a harder polymer. Silicone may increase patient comfort, but because of the softness of silicone, it is more difficult to guide the stent into the ureter. Once in the ureter, the softness of the silicone increases the likelihood of migration of the stent because rigid retention means are not available.

Thus, although stents have been designed to address one or more of the above problems specifically, there are currently no devices incorporating features that can be used to bypass most of the aforementioned disadvantages. It would thus be desirable to have a stent that provides one or more of the following attributes, easy insertion or implantation, strong retention, and increase patient comfort.

### BRIEF DESCRIPTION OF THE DRAWINGS

Like reference numbers indicate corresponding elements in the figures.

FIG. 1 is a front partial cross-sectional view of a ureteral stent within anatomy in accordance with an embodiment of the present invention;

FIGS. 2A and 2B are a partial cut-away view of the stent body portion in accordance with an embodiment of the present invention;

FIG. 3 is a partial cut-away view of the stent body portion in accordance with an embodiment of the present invention;

FIG. 4 is a partial cut-away view of the stent body portion in accordance with an embodiment of the present invention;

FIG. 5 is a partial cut-away view of the stent body portion in accordance with an embodiment of the present invention;

FIG. 6 is a partial cut-away view of the stent body portion in accordance with an embodiment of the present invention;

FIG. 7 is a partial cut-away view of the stent body portion in accordance with an embodiment of the present invention;

FIGS. 8A and 8B are partial cut-away views of the stent body portion in accordance with an embodiment of the present invention; and

FIG. 9 is a partial cut-away view of the body portion in accordance with another embodiment of the present invention.

### SUMMARY OF THE INVENTION

The present invention provides embodiments of medical devices that provide for fluid drainage while maintaining patient comfort.

An embodiment of the present invention relates to a stent comprising a tubular member at least a portion of which comprises a reinforcement structure. One advantage of this embodiment of the invention is, for example, that the reinforcement structure increases patient comfort by providing enhanced flexibility. A second advantage of this embodiment of the invention is, for example, that the reinforcement structure improves axial stiffness and radial stiffness allowing for a thinner tubular-member wall-thickness and larger lumen diameter.

An embodiment in accordance with the invention provides a stent comprising an elongated tubular member having a body portion, a proximate end portion and a distal end portion. The tubular member includes a wall and an axial lumen therein having a lumen surface. The tubular member further comprises a reinforcement structure extending at least a portion of a length of the body portion adjacent the distal end portion.

In accordance with an embodiment of the present invention, the reinforcement structure comprises a coiled filament embedded within a wall of the tubular member, the coiled filament adapted to provide a predetermined axial stiffness and radial stiffness to at least a portion of the body portion.

In accordance with another embodiment of the present invention, the reinforcement structure comprises a coiled filament adjacent to and in abutment with the lumen surface of the tubular member, the coiled filament adapted to provide a predetermined axial stiffness and radial stiffness.

In accordance with another embodiment of the present invention, the reinforcement structure comprises a plurality of rings and a plurality of longitudinal members. The longitudinal members are coupled to the plurality of rings that are spaced apart to define a tube-like structure. The reinforcement structure is embedded within the wall of the tubular member. The reinforcement structure is adapted to provide a predetermined axial stiffness and radial stiffness to at least a portion of the body portion.

In accordance with another embodiment of the present invention, the reinforcement structure comprises a plurality of rings and a plurality of longitudinal members. The longitudinal members are coupled to the plurality of rings that are spaced apart to define a tube-like structure. The reinforcement structure is adjacent to and in abutment with the lumen surface of the tubular member. The reinforcement structure is adapted to provide a predetermined axial stiffness and radial stiffness to at least a portion of the body portion.

In accordance with another embodiment of the present invention, the reinforcement structure comprises a cross-web member comprising a plurality of interconnected webs that bisect the lumen. The webs intersect at an axis of the lumen and extend radially to abut the lumen surface. The webs define a plurality of laterally running fluid passages suitable for providing a fluid path traversing the at least a portion of the body portion comprising the cross-web member.

In accordance with another embodiment of the present invention, the reinforcement structure comprises a cross-web member comprising a plurality of interconnected webs that bisect the lumen. The webs intersect at an axis of the lumen and extend radially to abut the lumen surface. The webs define a plurality of laterally running fluid passages suitable for providing a fluid path traversing the at least a portion of the body portion comprising the cross-web member. A tubular member further comprises a plurality of apertures adapted to provide fluid communication between an exterior of the tubular member and the fluid passages defined by the webs, the apertures substantially aligned with the fluid passages. A portion of the tubular member is disposed directly adjacent an edge of the webs defining a frame adapted to reduce any potential for trauma to any abutting body tissue.

### DETAILED DESCRIPTION

Reference will now be made to embodiments illustrated in the drawings and specific language which will be used to describe the same. It will nevertheless be understood that no limitation of the scope of the invention is thereby intended, such alterations and further modifications in the illustrated devices, as such further applications of the principles of the invention as illustrated therein as being contemplated as would normally occur to one skilled in the art to which the invention relates.

The invention relates to embodiments of medical devices (e.g., stents) for draining fluids. The invention increases patient comfort and prevents fluid retention if a stricture in a vessel develops. For simplicity and illustrative purposes, embodiments of the invention are described herein the context of draining urine from a kidney, through a ureter, and into the bladder. However, the invention is applicable to any situation that requires drainage within a body, from a body, and from one body structure to another. One such situation is, for example, biliary drainage from the gall bladder, through the biliary ducts, to the duodenum.

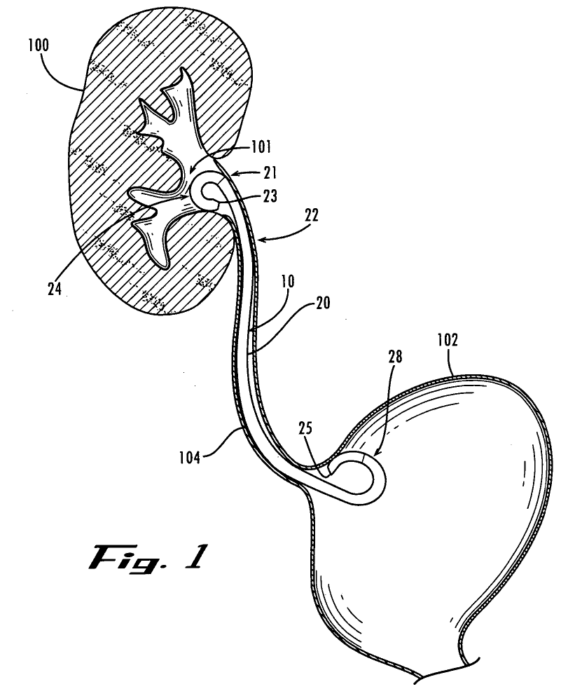

FIG. 1 is a front partial cross-sectional view of a ureteral stent 10 within anatomy in accordance with an embodiment of the present invention. The stent 10 comprises an elongated tubular member 20 having a relatively straight body portion 22 and opposed distal end portion 24 and proximal end portion 28. The distal end portion 24 is provided with means for retaining the distal end portion 24 in a kidney 100 and optionally, means for retaining the proximal end portion 28 in a bladder 102. The proximal end portion 28 of the stent 10 may have any of a variety of configurations providing a desired retaining effect or it may be entirely straight having no configuration for retention. The body portion 22 is adapted to be inserted into and reside substantially in the ureter 104. The distal end portion 24 has a predetermined configuration which advantageously serves several functions. The distal end portion 24 provides means for retaining the distal end portion 24 in the kidney 100. The distal end portion 24 is positioned to be angled from the body portion 22 in such a way to facilitate placement and removal of the stent 10 in a safe manner. The effect of the distal end portion 24 being positioned away from the body portion 22 of the stent 10 allows the distal end portion 24 to be partially uncoiled in the kidney 100 and for it to be removed without kinking or being pulled down intact into the ureter 104.

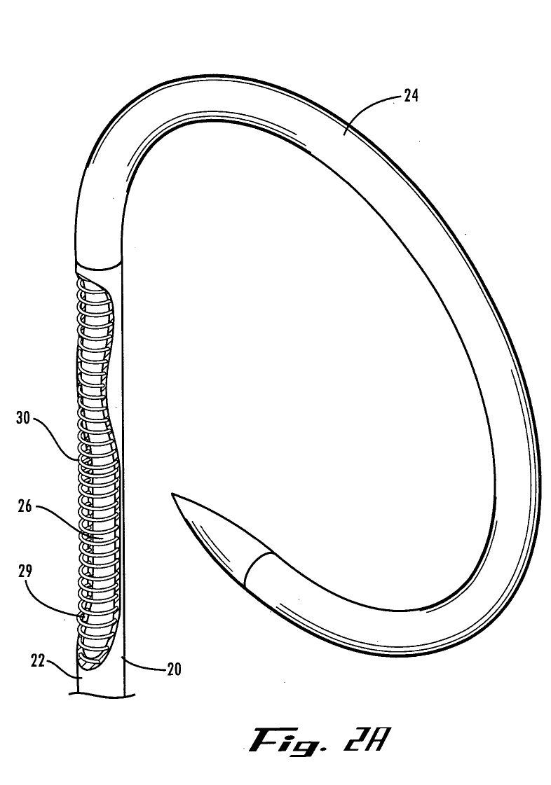

FIGS. 2A and 2B are partial cut-away views of the body portion 22 and distal end portion 24 in accordance with an embodiment of the present invention. The body portion 22 comprises an elongated tubular member 20 having a wall 29 and a length defining a lumen 26 therein. At least a portion of length of the tubular member 20 comprises an embedded reinforcement structure 30. The reinforcement member 30 is in a form of a coiled filament 32. The coiled filament 32 is embedded within the wall 29 of the tubular member 20.

The tubular member 20 may be formed from a variety of known materials which are biocompatible and have desired physical properties to be fabricated in the form hereafter described. Examples of suitable materials are silicone, thermoplastic material, elastomers, or any material known to one skilled in the art. The reinforcement member 30 is adapted to provide the tubular member 20 a predetermined axial and radial stiffness. Therefore, the tubular member 20 comprises a material that is relatively soft and compliant, which is more receptive to patient comfort.

The coiled filament 32 is provided with a predetermined hoop pitch suitable for the particular purpose to provide a predetermined radial and axial stiffness.

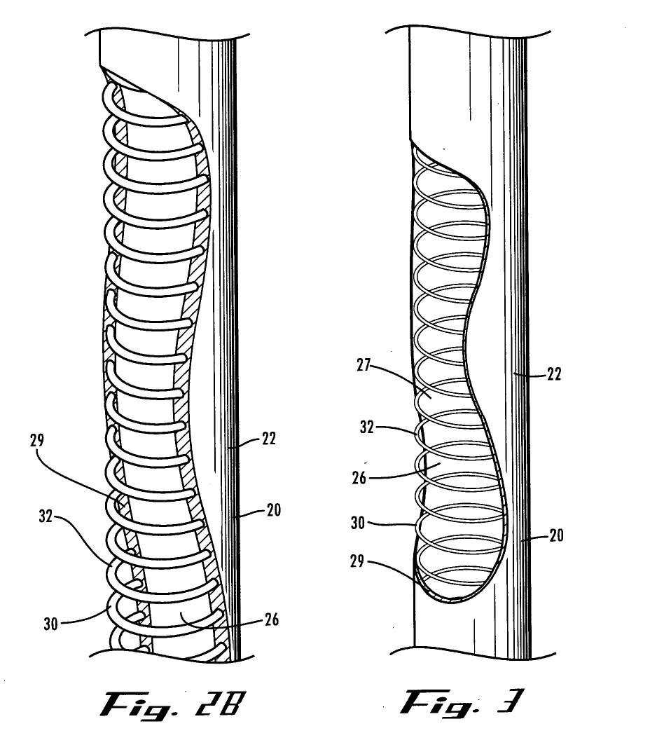

FIG. 3 is a partial cut-away view of the body portion 22 in accordance with another embodiment of the present invention. The body portion 22 comprises an elongated tubular member 20 having a wall 29 and a length defining a lumen 26 therein having a lumen surface 27. At least a portion of length of the tubular member 20 comprises a reinforcement structure 30 adjacent the lumen surface 27. The reinforcement structure 30 is in a form of a coiled filament 32.

Similarly, the reinforcement member 30 is adapted to provide the tubular member 20 axial stiffness and radial stiffness. Therefore, the tubular member 20 comprises a material that is relatively soft and compliant, which is more receptive to patient comfort.

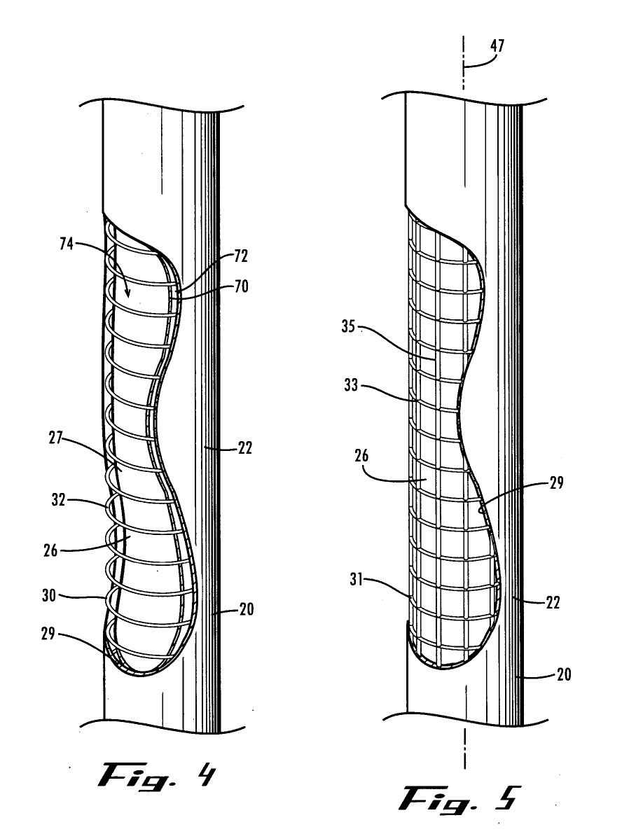

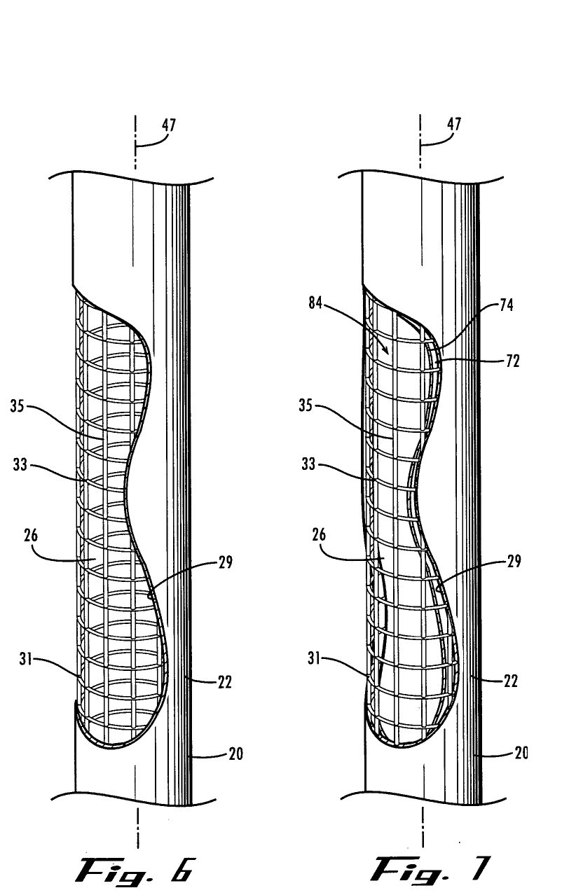

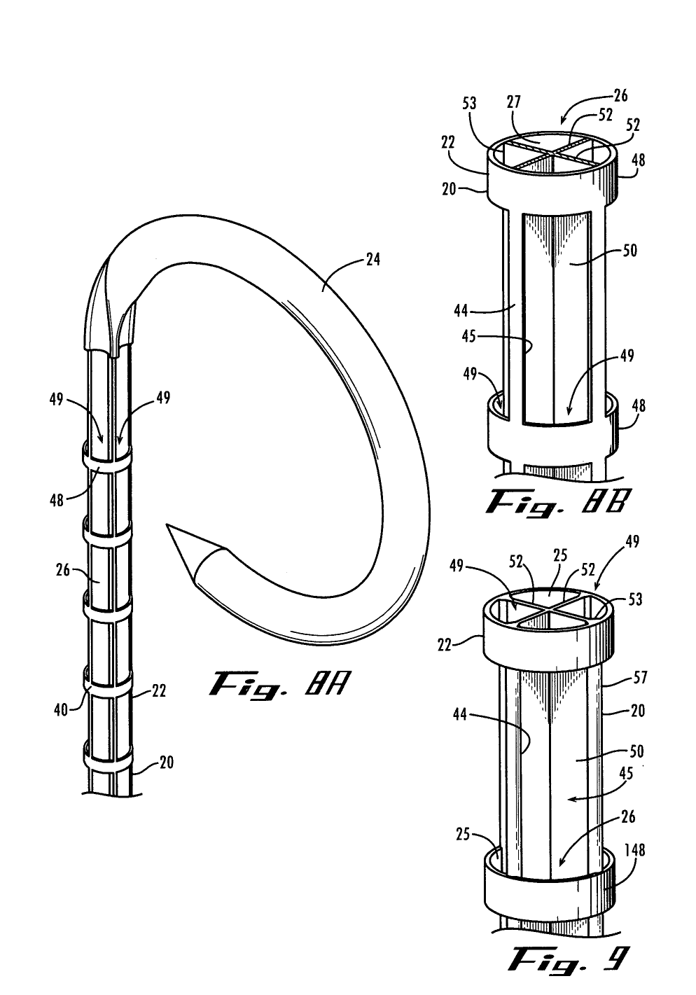

FIG. 4 is a partial cut-away view of a body portion 22 in accordance with another embodiment of the present invention. The body portion 22 comprises an elongated tubular member 20 having a wall 29 and a length defining a lumen 26 therein having a lumen surface 27. At least a portion of length of the tubular member 20 comprises a reinforcement structure 30 adjacent the lumen surface 27. The reinforcement structure 30 is in a form of a coiled filament 32. The coiled filament 32 defines a coil lumen 74. A second tubular member 70 is positioned within the coil lumen 74, wherein the reinforcement structure 30 is freely movable between the tubular member 20 and second tubular member 70. Because the reinforcement structure 30 is not coupled to either the tubular member 20 or second tubular member 70, and freely movable relative to the same, the body portion 22 is provided with a high degree of flexibility, compressibility, and expandability while retaining radial strength.

FIG. 5 is a partial cut-away view of the body portion 22 in accordance with another embodiment of the present invention. The body portion 22 comprises an elongated tubular member 20 having a length defining a lumen 26 therein. At least a portion of length of the tubular member 20 comprises an embedded reinforcement structure 31. The reinforcement structure 31 is in a form of a plurality of rings 33 coupled with longitudinal members 35 that run substantially parallel with a long axis 47 of the lumen 26. The reinforcement structure 31 is embedded within the wall 29 of the tubular member 20.

FIG. 6 is a partial cut-away view of the body portion 22 in accordance with another embodiment of the present invention. The body portion 22 comprises an elongated tubular member 20 having a length defining a lumen 26 therein having a lumen surface 27. At least a portion of length of the tubular member 20 comprises a reinforcement structure 31. The reinforcement structure 31 is in a form of a plurality of rings 33 coupled with longitudinal members 35 that run substantially parallel with the axis 47 of the lumen 26. The reinforcement structure 31 is adjacent the lumen 26 surface.

In the embodiments of FIGS. 4 and 5, the plurality of rings 33 provide a predetermined radial stiffness to the tubular member 20. The longitudinal members 35 provide a predetermined axial stiffness to the tubular member 20. The rings 33 are shown in FIGS. 4 and 5 as substantially equally spaced along the axial direction. The rings 33 are shown by way of example and, in other embodiments, are not necessarily equally spaced in the axial direction. Similarly, the longitudinal members 35 are shown to be substantially equally spaced around the diameter of the tubular member 20. The longitudinal members 35 are shown by way of example and, in other embodiments, are not necessarily equally spaced around the diameter of the tubular member 20.

The rings 33 and longitudinal members 35 are formed into a tubular mesh-like configuration. Known manufacturing methods include, but are not limited to, welding and weaving. The tubular mesh-like configuration of the reinforcement structure 31 comprises an outer diameter substantially the same as the diameter of the lumen 26.

FIG. 7 is a partial cut-away view of the body portion 22 in accordance with another embodiment of the present invention. The body portion 22 comprises an elongated tubular member 20 having a length defining a lumen 26 therein having a lumen surface 27. At least a portion of length of the tubular member 20 comprises a reinforcement structure 31. The reinforcement structure 31 is in a form of a plurality of rings 33 coupled with longitudinal members 35 that run substantially parallel with the axis 47 of the lumen 26. The reinforcement structure 31 is adjacent the lumen 26 surface.

The reinforcement structure 31 defines a reinforcement lumen 84. A second tubular member 70 is positioned within the reinforcement lumen 84, wherein the reinforcement structure 31 is freely movable between the tubular member 20 and second tubular member 70. Since the reinforcement structure 31 is not coupled to either the tubular member 20 nor second tubular member 70, and freely movable relative to the same, the body portion 22 is provided with a high degree of flexibility, while retaining radial strength.

FIGS. 8A and 8B are perspective and partial cut-away views, respectively, of the body portion 22 in accordance with another embodiment of the present invention. The body portion 22 comprises an elongated tubular member 20 having a length defining a lumen 26 therein having a lumen surface 27. At least a portion of length of the tubular member 20 comprises a cross-web member 50 adjacent the lumen surface 43. The cross-web member 50 comprises a plurality of interconnected webs 52 that bisect the lumen 26. The webs 52 of the cross-web member 50 intersect at the axis 47 of the lumen 26 and extend radially to abut the lumen surface 25. The webs 52 define a plurality of laterally running fluid passages 49 suitable for providing a fluid path traversing the portion of the body portion 22 comprising the cross-web member 50.

The webs 52 are adapted to provide a relatively high degree of radial stiffness to the body portion 22 while maintaining relative longitudinal flexibility. The webs 52 comprise a material suitable for the particular purpose, such as, but not limited to, stainless steel or nickel titanium alloy. Certain alloys of nickel titanium, known as Nitinol, can be provided with a super-elastic physical property. A super-elastic property provides a high degree of flexibility without elastic deformation, which is a desired property for the particular purpose.

At least a portion of the length of the body portion 22 comprising the cross-web member 50 further comprises a plurality of apertures 45. The apertures 45 are adapted to provide fluid communication between exterior of the tubular member 20 and the fluid passages 49 defined by the webs 52. The apertures 45 are substantially aligned with the fluid passages 49. A portion of the tubular member 20 is disposed directly adjacent an edge 53 of the webs 52 defining a frame 44 adapted to greatly reduce the risk for trauma to any abutting body tissue. The apertures 45 define a plurality of annular rings 48 that provide a predetermined radial stiffness to the tubular member 20. Further, the plurality of annular rings 48 provide a means to retain a guidewire (not shown) or the like to within the lumen 26, such as, for deployment.

FIG. 9 is a partial cut-away view of the body portion 22 in accordance with another embodiment of the present invention. The body portion 22 comprises a plurality of individual rings 148 disposed along the length of and around the diameter defined by the edges 53 of the cross-web member 50 and coupled thereto. A suitable coating 57 is applied to the edge 53 rather than a portion of the tubular member 20 disposed directly adjacent an edge 53 of the webs 52.

Referring again to FIG. 1, the stent 10 includes a body portion 22 adapted to be positioned within the ureter 104. The stent 10 thereby provides fluid communication along its length from the distal end portion 24 to the proximal end portion 28. The central lumen 26 may extend fully to a proximal tip 25 and distal tip 23 although it is not essential as long as fluid communication between the kidney 100 and bladder 102 is maintained through the lumen 26. When the stent 10 is placed within the ureter 104 with the proximal end portion 28 in the usual fashion, further communication is provided by means of the central lumen 26 and when provided, end openings at the proximal end portion 28 and distal end portion 24.

The proximal end portion 28 is provided with retention means for retaining the proximal end portion 28 in the bladder 102, or alternatively, it may be free of retention means to serve as a drain. If a retention means is utilized, it may assume a variety of forms such as those commonly used in the art. For example, the proximal end portion 28 may include a J-shaped curve.

The stent 10 also includes a distal end portion 24 which includes retention means for retaining the distal end portion 24 in the kidney 100. The retention means at the distal end portion 24 comprises a distal end portion 24 being set in the shape in a variety of forms such as those commonly used in the art.

The distal end portion 24 joints the body portion 22 of the stent 10 at a transition section 21. The placement of the stent 10 in a kidney 100 is shown in FIG. 1. The distal end portion 24 is placed in the renal cavity 101 with the distal end portion 24 positioned away from the ureteropelvic junction. The curvature of the distal end portion 24 is adapted for retention of the distal end portion 24 in the kidney 100. The configuration of the distal end portion 24 of the stent 10 permits easy placement of the distal end portion 24 in the renal pelvis.

Once a physician has selected a stent 10 for use in the patient, after considering the normal size of the ureteral passage and the length of the ureter 104, the placement of the ureteral stent 10 may by accomplished in accordance with known prior art techniques. A wire stylet, or guidewire (not shown), which can be formed of stainless steel, is inserted into the proximal tip 25 of the proximal end portion 28 of the stent 10 and into the lumen 26 and advanced into the distal end portion 24.

The stent 10 is drawn over the guidewire to straighten the proximal end portion 28 and the curved distal end portion 24. The guidewire is inserted up to, but not beyond, the distal tip 23 and can be immobilized in the stent 10 by any suitable known locking means, not shown.

Confirmation that the renal pelvis has been entered by the stent 10 can be obtained by x-ray. If desired, radiopaque measurement markings or other suitable radiopaque indicia can be incorporated on the stent 10 and are visible during x-ray examination to aid in confirming the position of the stent 10.

After the stent 10 has been inserted into a predetermined distance into the renal pelvis, the guidewire is withdrawn, enabling the curl to form in the distal end portion 24. The distal end portion 24 bears against the walls of the renal cavity 101 and ureter 104.

Alternatively, the ureteral stent 10 may be placed in the ureter 104 during surgery.

Dimensions for the ureteral stent 10 of the present invention are not critical; however, they include internal diameters of 4.5 to 8.5 French, and lengths ranging from 20 cm. to 32 cm.

The distal end portion 24 transitions to the body portion 22 at the transition section 21. The body portion 22 generally resides within the ureter. Also, the proximal end portion 28 extends out of the ureter 104 and into the bladder 102. For example, the stent 10 of FIG. 1 is positioned in the manner described above.

In one embodiment in accordance with the present invention, the transition section 21 between the body portion 22 and the distal end portion 24 is characterized by single-piece construction. A single piece of material forms the distal end portion 24 and the body portion 22. In alternative embodiments, two or more pieces of one or more materials may be joined at the transition section 21 to form the stent 10 in accordance with the invention. Additionally, the body portion 22 remains substantially constant in cross-sectional area along its length up to and including the transition portion 21 between the body portion 22 and the distal end portion 24. The transition between the body portion 22 and the proximate end portion 28 is substantially as described for the distal end portion 24.

In certain embodiments of medical devices according to the invention, the body portion 22 may have, for example, a circular cross-section. The circular cross-section may be of any circumference that is of suitable size for the body structure into which the device is placed. For example, for use in the kidney 100, ureter 104, and bladder 102, the body portion 22 may be from about 8.0 French to about 4.8 French in size. The stent 10 may have a substantially constant cross-sectional area along its length. Alternatively, the stent 10 may be tapered from about the distal end portion 24 to about the proximal end portion 28 with the size of the section decreasing from about 7 French to about 3 French. Other French sizes and tapering configurations are useful depending upon either the average size in a population or the actual size in an individual of the body structure or structures into which the device is placed.

The tubular member 20 according to the invention may be constructed from any of a number of materials. Those materials that are useful include, for example, materials that are able to flex but also retain their shape, to a degree, when they are perturbed. Additionally, useful materials are, for example, materials that have a resilient quality, being able to regain at least some of their original shape when the stent 10 ceases to be perturbed and/or resist, for example, compression. One such material that combines these features is Percuflex™. Moreover, thermo-formable materials, including, for example, Percuflex™ are useful in the practice of the invention.

In operation, the distal end portion 24 of the stent 10 is inserted through the bladder 102 and ureter 104 into the kidney 100. The distal end portion 24 may be straightened for ease of insertion. One manner to straighten the stent 10 is to produce relative movement between a straightening device (e.g., a sheath, not shown) and the distal end portion 24, such that the straightening device moves distally relative to the distal end portion 24, thereby uncurling the distal end portion 24 to the straightened position. Once at least some portion of the distal end portion 24 is positioned within the kidney 100, the straightening device is removed. Thus, once the straightening device is withdrawn, the distal end portion 24 in a straightened position returns to its curled shape.

An alternative method to straighten the curl of the distal end portion 24 is to use a guidewire which slides within the lumen 26 of the stent 10 and is sufficiently stiff to hold the curl in a straight configuration (e.g., the distal end portion 24 in a straightened position). Relative movement is produced between the guidewire and the distal end portion 24, such that the guidewire moves distally relative to the distal end portion 24, thereby uncurling the distal end portion 24 to the straightened position. Once at least a portion of the distal end portion 24 is positioned within the kidney 100, the guidewire is withdrawn. Other modes of inserting and/or straightening a device also known in the art.

The distal end portion 24 is flexible, yet sufficiently rigid to be self-supporting rather than pliable so as to provide sufficient anchor in the vessel. Over most of its length, distal end portion 24 has a diameter substantially equal to the diameter of the body portion 22, with the distal tip 23 forming a converging end. Alternatively, the distal end portion 24 may have any number of suitable cross-sections for the particular purpose.

Identical concerns that are mentioned above with respect to stents are also applicable in the catheter and intubation arts, which include, without limitation: intravenous catheters, guiding catheters, sheaths, umbilical catheters, trocar catheters, heart catheters (including valvostomy catheters, angioplasty catheters, arthroscopy catheters, and the like), perfusion catheters, suction catheters, oxygen catheters, endoscopy catheters, endotracheal tubes, stomach tubes, feeding tubes, lavage tubes, rectal tubes, urological tubes, irrigation tubes, aneurysm shunts, stenosis dialators, trocars, and inserters, generally.

While the invention has been described in connection with specific embodiments thereof, it will be understood that it is capable of further modification, and this application is intended to cover any variations, uses, or adaptations of the invention following, in general, the principles of the invention and including such departures from the present disclosure as come within known or customary practice in the art to which the invention pertains and as may be applied to the essential features hereinbefore set forth, and as fall within the scope of the invention and the limits of the appended claims.

1. A stent comprising:
an elongated tubular member having a body portion, a proximate end portion and a distal end portion, the tubular member having a wall and an axial lumen therein having a lumen surface; and
a reinforcement structure extending at least a portion of a length of the body portion adjacent the distal end portion.

2. The stent of claim 1, where the reinforcement structure comprises:
a coiled filament embedded within the wall of the tubular structure, the coiled filament adapted to provide a predetermined axial stiffness and radial stiffness.

3. The sent of claim 1, where the reinforcement structure comprises:
a coiled filament adjacent to and in abutment with the lumen surface of the tubular structure, the coiled filament adapted to provide a predetermined axial stiffness and radial stiffness.

4. The stent of claim 1, where the reinforcement structure comprises:
a plurality of rings; and
a plurality of longitudinal members, the longitudinal members coupled to the plurality of rings spaced apart to define a tube-like structure, the reinforcement structure embedded within the wall of the tubular structure, the reinforcement structure adapted to provide a predetermined axial stiffness and radial stiffness to at least a portion of the body portion.

5. The stent of claim 1, where the reinforcement structure comprises:
a plurality of rings; and
a plurality of longitudinal members, the longitudinal members coupled to the plurality of rings spaced apart to define a tube-like structure, the reinforcement structure adjacent to and in abutment with the lumen surface of the tubular structure, the reinforcement structure adapted to provide a predetermined axial stiffness and radial stiffness to at least a portion of the body portion.

6. The stent of claim 1, where the reinforcement structure comprises:
a cross-web member comprising a plurality of interconnected webs that bisect the lumen, the webs intersect at an axis of the lumen and extend radially to abut the lumen surface, the webs defining a plurality of laterally running fluid passages suitable for providing a fluid path traversing the at least a portion of the body portion comprising the cross-web member.

7. The stent of claim 6, further comprising:
a plurality of apertures adapted to provide fluid communication between an exterior of the tubular member and the fluid passages defined by the webs, the apertures substantially aligned with the fluid passages; and
a portion of the tubular member disposed directly adjacent an edge of the webs defining a frame adapted to reduce any potential for trauma to any abutting body tissue.

8. The stent of claim 7, wherein the apertures define a plurality of annular rings adapted to retain a guide within one of the fluid passages.

9. The stent of claim 1, wherein the distal end portion comprises a retaining structure.

10. The stent of claim 1, wherein the stent comprises a ureteral drainage stent.

11. A ureteral stent for maintaining drainage between the kidney and bladder comprising:
an elongated flexible tubular member having proximal end portion and a distal end portion coupled by a body portion having an axis including a central lumen to provide fluid communication between the proximal end portion and distal end portion, the proximal end portion adapted for placement in the bladder, the distal end portion including retention means adapted for retaining the distal end portion in the kidney, at least a portion of the body portion comprising a reinforcement structure extending at least a portion of a length of the body portion adjacent the distal end portion.

12. The ureteral stent of claim 11, where the reinforcement structure comprises:
a coiled filament embedded within the wall of the tubular structure, the coiled filament adapted to provide a predetermined axial stiffness and radial stiffness.

13. The sent of claim 11, where the reinforcement structure comprises:
a coiled filament adjacent to and in abutment with the lumen surface of the tubular structure, the coiled filament adapted to provide a predetermined axial stiffness and radial stiffness.

14. The stent of claim 11, where the reinforcement structure comprises:
a plurality of rings; and
a plurality of longitudinal members, the longitudinal members coupled to the plurality of rings spaced apart to define a tube-like structure, the reinforcement structure embedded within the wall of the tubular structure, the reinforcement structure adapted to provide a predetermined axial stiffness and radial stiffness to at least a portion of the body portion.

15. The stent of claim 11, where the reinforcement structure comprises:
a plurality of rings; and
a plurality of longitudinal members, the longitudinal members coupled to the plurality of rings spaced apart to define a tube-like structure, the reinforcement structure adjacent to and in abutment with the lumen surface of the tubular structure, the reinforcement structure adapted to provide a predetermined axial stiffness and radial stiffness to at least a portion of the body portion.

16. The stent of claim 11, where the reinforcement structure comprises:
a cross-web member comprising a plurality of interconnected webs that bisect the lumen, the webs intersect at an axis of the lumen and extend radially to abut the lumen surface, the webs defining a plurality of laterally running fluid passages suitable for providing a fluid path traversing the at least a portion of the body portion comprising the cross-web member.

17. The stent of claim 16, further comprising:
a plurality of apertures adapted to provide fluid communication between an exterior of the tubular member and the fluid passages defined by the webs, the apertures substantially aligned with the fluid passages; and
a portion of the tubular member disposed directly adjacent an edge of the webs defining a frame adapted to reduce any potential for trauma to any abutting body tissue.

18. The stent of claim 17, wherein the apertures define a plurality of annular rings adapted to retain a guide within one of the fluid passages.

19. The stent of claim 11 wherein the distal end portion comprises a retaining structure.

20. The stent of claim 11 wherein the stent comprises a ureteral drainage stent.

21. A method for placing a stent comprising:
inserting a body portion into a ureter, a distal end portion into a kidney and a proximal end portion into a bladder, the stent comprising:
an elongated flexible tubular member having proximal end portion and a distal end portion coupled by a body portion having an axis including a central lumen to provide fluid communication between the proximal end portion and distal end portion, the proximal end portion adapted for placement in the bladder, the distal end portion including retention means adapted for retaining the distal end portion in the kidney, at least a portion of the body portion comprising a reinforcement structure extending at least a portion of a length of the body portion adjacent the distal end portion.

22. The method of claim 21, further comprising:
inserting a guidewire into the lumen of the stent, wherein the reinforcement structure comprises:
a coiled filament embedded within the wall of the tubular structure, the coiled filament adapted to provide a predetermined axial stiffness and radial stiffness.

23. The method of claim 21, further comprising:
inserting a guidewire into the lumen of the stent, wherein the reinforcement structure comprises:
a coiled filament adjacent to and in abutment with the lumen surface of the tubular structure, the coiled filament adapted to provide a predetermined axial stiffness and radial stiffness.

24. The method of claim 21, further comprising:
inserting a guidewire into the lumen of the stent, wherein the reinforcement structure comprises:
a plurality of rings; and
a plurality of longitudinal members, the longitudinal members coupled to the plurality of rings spaced apart to define a tube-like structure, the reinforcement structure embedded within the wall of the tubular structure, the reinforcement structure adapted to provide a predetermined axial stiffness and radial stiffness to at least a portion of the body portion.

25. The method of claim 21, further comprising:
inserting a guidewire into the lumen of the stent, wherein the reinforcement structure comprises:
a plurality of rings; and
a plurality of longitudinal members, the longitudinal members coupled to the plurality of rings spaced apart to define a tube-like structure, the reinforcement structure adjacent to and in abutment with the lumen surface of the tubular structure, the reinforcement structure adapted to provide a predetermined axial stiffness and radial stiffness to at least a portion of the body portion.

26. The method of claim 21, further comprising:
inserting a guidewire into the lumen of the stent, wherein the reinforcement structure comprises:
a cross-web member comprising a plurality of interconnected webs that bisect the lumen, the webs intersect at an axis of the lumen and extend radially to abut the lumen surface, the webs defining a plurality of laterally running fluid passages suitable for providing a fluid path traversing the at least a portion of the body portion comprising the cross-web member.

27. The method of claim 25, further comprising:
inserting a guidewire into the lumen of the stent, wherein the reinforcement structure comprises:
a plurality of apertures adapted to provide fluid communication between an exterior of the tubular member and the fluid passages defined by the webs, the apertures substantially aligned with the fluid passages; and
a portion of the tubular member disposed directly adjacent an edge of the webs defining a frame adapted to reduce any potential for trauma to any abutting body tissue.

28. The method of claim 26, wherein the apertures define a plurality of annular rings adapted to retain the guide within one of the fluid passages, the method further comprising:
inserting the guidewire into one of the fluid passages.

29. The method of claim 21 wherein the distal end portion comprises a retaining structure, the method further comprising:
inserting the guidewire into the distal end portion.
